(19) United States
(12) Patent Application Publication
(10) Pub. No.: US 2008/0279938 A1
(43) Pub. Date: Nov. 13, 2008

(54) ZALTOPROFEN-CONTAINING SUSTAINED RELEASE TABLET AND PROCESS FOR THE PREPARATION THEREOF

(75) Inventors: CHEONG WEON CHO, SEONGNAM-SI (KR); JEONG KU, YONGIN-SI (KR); HEE CHOL KANG, DAEJEON (KR); QING Ri CAO, SUWON-SI (KR); EUN YOUNG YANG, YONGIN-SI (KR); TAE KUN AN, YONGIN-SI (KR); EUN KYUNG JEON, YONGIN-SI (KR); JAE KYOUNG KO, YONGIN-SI (KR); HEA RAN SUH, ICHEON-SI (KR); HYE JIN HAN, SUWON-SI (KR); GANG SOO CHAE, GUNSAN-SI (KR)

Correspondence Address: KNOBBE MARTENS OLSON & BEAR LLP, 2040 MAIN STREET, FOURTEENTH FLOOR, IRVINE, CA, 92614, US

(73) Assignee: CJ CHEILJEDANG CORPORATION, SEOUL (KR)

(21) Appl. No.: 12/181,112

(22) Filed: Jul. 28, 2008

Related U.S. Application Data
(63) Continuation of application No. PCT/KR2007/000441, filed on Jan. 25, 2007.

(30) Foreign Application Priority Data
Jan. 27, 2006 (KR) 10-2006-0009058

(51) Int. Cl. A61K 9/26; A61K 31/38; A61P 29/00

(52) U.S. Cl. 424/470; 514/431

(57) ABSTRACT

Disclosed is a zaltoprofen-containing sustained release tablet comprising granules containing zaltoprofen and a binder, in which the granules are dispersed in a matrix comprising a hydrophilic polymer, and a diluent is present either in the granules or in the matrix.

### CROSS-REFERENCE TO RELATED PATENT APPLICATIONS

This application is a continuation application under 35 U.S.C. § 365(c) of International Application No. PCT/KR2007/000441, filed Jan. 25, 2007 designating the United States. International Application No. PCT/KR2007/000441 was published in English as WO2007/086694 A1 on Aug. 2, 2007. This application further claims the benefit of the earlier filing date under 35 U.S.C. § 365(b) of Korean Patent Application No. 10-2006-0009058 filed Jan. 27, 2006. This application incorporates herein by reference the International Application No. PCT/KR2007/000441 including the International Publication No. WO2007/086694 A1 and the Korean Patent Application No. 10-2006-0009058 in their entirety.

### BACKGROUND

1. Field

The present disclosure relates to a drug delivery system, and more particularly, to a sustained release oral formulation.

2. Discussion of the Related Technology

Zaltoprofen is a non-steroidal anti-inflammatory drug, and has excellent effects even on post-surgery or post-trauma chronic inflammation. Zaltoprofen is needed to be administered typically three times a day in a dose of about 80 mg, for adults. Therefore, in order to improve patient's convenience and dosage compliance, and to reduce gastrointestinal side effects, an once-daily dosage formulation which can be administered only once a day is desirable.

An oral sustained release delivery system which is intended to control the release of the active ingredient drug so that the drug can be administered only once a day, can be prepared in the form of a matrix comprising a polymer. The active ingredient may be slowly released in the gastrointestinal tract by means of decomposition of the matrix and diffusion of the active ingredient in the matrix. The sustained release drug dosage form including such matrix system is usually prepared in the form of a compressed tablet.

The technology of using a matrix of a hydrophilic polymer among many polymers for the purpose of sustained release of a drug, is known in the art. In particular, a sustained release tablet comprising a matrix of hydroxypropylmethylcellulose among hydrophilic polymers is well known in the art of pharmaceutics.

U.S. Pat. No. 3,065,143 discloses the use of a specific hydrophilic rubber containing hydroxypropylmethylcellulose in the preparation of a sustained release tablet.

Meanwhile, U.S. Pat. No. 3,458,622 discloses a process for preparing a sustained release tablet using a combination of povidone and carbomer. U.S. Pat. No. 4,389,393 discloses a sustained release therapeutic composition based on a matrix comprising high molecular weight hydroxypropylmethylcellulose.

However, there has been no report to date on sustained release tablets of zaltoprofen which enables administration of once-daily.

The foregoing discussion in the background section is to provide general background information, and does not constitute an admission of prior art.

### SUMMARY

One aspect of the invention provides a zaltoprofen-containing sustained release tablet comprising granules containing zaltoprofen and a binder, wherein the granules are dispersed in a matrix comprising a hydrophilic polymer, wherein the tablet further comprises a diluent that is present in the granules, in the matrix.

In the foregoing tablet, 5 to 60 parts by weight of the hydrophilic polymer, 1 to 30 parts by weight of the binder, and 5 to 60 parts by weight of the diluent may be contained in the tablet, relative to 100 parts by weight of zaltoprofen. The hydrophilic polymer may have a viscosity of 5 to 100,000 cps. The binder may be selected from the group consisting of povidone, gelatin, hydroxypropylcellulose, hydroxypropylmethylcellulose, and an enteric polymer. The diluent may be selected from the group consisting of lactose, dextrin, starch, crystalline cellulose, an enteric polymer, calcium hydrogen phosphate, calcium carbonate, sugars and silicon dioxide. The enteric polymer may be selected from the group consisting of Eudragit L, Eudragit S, Eudragit L-100-55-Rohm Pharma, Eudragit L30D-Rohm Pharma, cellulose acetate phthalate, polyvinyl acetate phthalate, and hydroxypropylmethylcellulose phthalate. The enteric polymer may be selected from the group consisting of Eudragit L, Eudragit S, Eudragit L-100-55-Rohm Pharma, Eudragit L30D-Rohm Pharma, cellulose acetate phthalate, polyvinyl acetate phthalate, and hydroxypropylmethylcellulose phthalate.

Another aspect of the invention provides a method of preparing the foregoing sustained release tablet. The method comprising: providing granules containing zaltoprofen and the binder; mixing the granules with a hydrophilic polymer and pharmaceutically acceptable additives so as to provide a mixture; and shaping the mixture into a tablet. In the foregoing method, the diluent may be contained in the granules. Mixing may comprise adding the diluent so as to include the diluent in the mixture.

Still another aspect of the invention provides a method of treating a patient in need of zaltoprofen. The method comprises: providing the foregoing zaltoprofen-containing sustained release tablet; and administering the tablet to a patient. In the foregoing method, administering the tablet may be less frequent than twice a day. Administering the tablet may be once daily.

Accordingly, an aspect of the present invention to provide a sustained release tablet of zaltoprofen. Another aspect of the invention to provide a method for preparing a sustained release tablet of zaltoprofen.

One aspect of the present invention provides a zaltoprofen-containing sustained release tablet comprising granules containing zaltoprofen and a binder that are dispersed in a matrix containing a hydrophilic polymer, and a diluent that is present either in the granules or in the matrix.

The hydrophilic polymer, binder and diluent contained in the sustained release tablet may be present in the amounts of 5 to 60 parts by weight of the hydrophilic polymer, 1 to 30 parts by weight of the binder, and 5 to 60 parts by weight of the diluent, relative to 100 parts by weight of zaltoprofen. The hydrophilic polymer which is the constituent component of the matrix preferably has a viscosity of 5 to 100,000 cps.

The hydrophilic polymer may be selected from the group consisting of, for example, acacia gum, tragacanth gum, locust bean gum, guar gum, Karaya gum, agar-agar, pectin, carrageenan, soluble or insoluble alginates, methylcellulose, hydroxypropylmethylcellulose, carbomer, hydroxypropylcellulose, hydroxyethylcellulose, sodium carboxymethylcellulose, carboxypolymethylene, gelatin, casein, zein, bentonite, a natural or synthetic, anionic or nonionic hydrophilic rubber, modified cellulose-based materials, and proteinaceous materials, but the invention is not limited thereto.

The binder forming the granules may be selected from the group consisting of povidone, gelatin, hydroxypropylcellulose, hydroxypropylmethylcellulose, and an enteric polymer. The diluent distributed in the granules or matrix of the sustained release tablet may be selected from the group consisting of lactose, dextrin, starch, crystalline cellulose, an enteric polymer, calcium hydrogen phosphate, calcium carbonate, sugars, and silicon dioxide. The enteric polymer that can be used as the binder or diluent may be selected from the group consisting of Eudragit L, Eudragit S, Eudragit L-100-55-Rohm Pharma, Eudragit L30D-Rohm Pharma, cellulose acetate phthalate, polyvinyl acetate phthalate, and hydroxypropylmethylcellulose phthalate.

Another aspect of the invention provides a method for preparing a zaltoprofen-containing sustained release tablet, comprising: preparing granules containing zaltoprofen and a binder; and tabletting the mixture after mixing the granules with a hydrophilic polymer and pharmaceutically acceptable additives, in which a diluent is added during the step of preparing the granules or during the step of tabletting.

### BRIEF DESCRIPTION OF THE DRAWINGS

FIG. 1 is a graph showing the dissolution rate over time, measured in a dissolution test of the sustained release tablets prepared in Examples 1 to 3 of the present invention.

FIG. 2 is a graph showing the dissolution rate over time, measured in a dissolution test of the sustained release tablet prepared in Example 4 of the invention.

FIG. 3 is a graph showing the dissolution rate over time, measured in a dissolution test of the sustained release tablets prepared in Examples 5 to 7 of the invention.

FIG. 4 is a graph showing the dissolution rate over time, measured in a dissolution test of the sustained release tablets prepared in Examples 8 to 10 of the invention.

### DETAILED DESCRIPTION OF EMBODIMENTS

Hereinafter, various embodiment will be described in more detail.

The disclosure relates to a zaltoprofen-containing sustained release tablet which enables once-daily administration of zaltoprofen, a drug exhibiting excellent anti-inflammatory effect as well as analgesic effect, thus increasing patient's dosage compliance and convenience. It was not expected to obtain a sustained release tablet which can release zaltoprofen at a desired constant rate, such as a desired dissolution rate, only with the hydrophilic polymers that have been predominantly used in the preparation of conventional sustained release tablets. Thus, the inventors introduced a binder and a diluent to a sustained release tablet so as to facilitate the control of the release of zaltoprofen. Furthermore, since zaltoprofen is a sparingly soluble drug, and has tabletting problems such as capping when provided in a fine powder form during tabletting, the inventors prepared granules of the drug to improve the fluidity and to facilitate tabletting.

Therefore, the zaltoprofen-containing sustained release tablet according to an embodiment of the present invention has granules containing zaltoprofen and a binder, dispersed in a matrix comprising a hydrophilic polymer, and a diluent is homogeneously present in the granules or the matrix. Such sustained tablet can result in stable sustained release at a desired rate, due to the binder present in the granules, and the diluent present in the granules or matrix, in addition to the hydrophilic polymer.

The proportions of the constitutional components in the sustained release tablet are preferably 5 to 60 parts by weight of the hydrophilic polymer, 1 to 30 parts by weight of the binder, and 5 to 60 parts by weight of the diluent, relative to 100 parts by weight of zaltoprofen. If the contents of the hydrophilic polymer and the binder exceed the ranges mentioned above, the release of the active ingredient becomes so slow that it would be difficult to reach the desired blood concentration of the active ingredient. If the contents of the hydrophilic polymer and the binder are less than the mentioned ranges, the effect of sustained release enabling once-daily administration cannot be expected. In the case of the diluent, if the content of the diluent exceeds the range mentioned above, there is a problem that the active ingredient is rapidly released, even faster than the desired rate of sustained release.

The hydrophilic polymer that can be used for an embodiment of the invention is a hydrophilic polymer having a viscosity ranging from 5 to 100,000 cps. If the viscosity is higher than the range, the drug release becomes so slow and cannot be achieved at a desired rate.

The hydrophilic polymer that is suitable to be used as the hydrophilic polymer constituting the matrix of the sustained release tablet according to an embodiment of the invention, may be selected from the group consisting of, for example, acacia gum, tragacanth gum, locust bean gum, guar gum, Karaya gum, agar-agar, pectin, carrageenan, soluble or insoluble alginates, methylcellulose, hydroxypropylmethylcellulose, carbomer, hydroxypropylcellulose, hydroxyethylcellulose, sodium carboxymethylcellulose, carboxypolymethylene, gelatin, casein, zein, bentonite, a natural or synthetic, anionic or nonionic hydrophilic rubber, modified cellulose-based materials, and proteinaceous materials, but the invention is not limited thereto.

Among these hydrophilic polymers, hydroxypropylmethylcellulose (e.g., Methocel™, Dow Chemical) or carbomer (e.g., Carbopol™, Noveon, Inc.) can be preferably used.

The hydrophilic polymer may be contained in an amount of 5 to 60 parts by weight, preferably 15 to 40 parts by weight, relative to 100 parts by weight of the active ingredient.

Zaltoprofen is present in the sustained release tablet in the form of granules with a binder, and zaltoprofen is formed into granules together with the binder as such, in order to facilitate the tabletting step and to facilitate dissolution of the active ingredient, thereby facilitating the control of the release of the active ingredient. The binder used in the granulation may be any binder that is conventionally used in formulating solid oral preparations, and examples thereof include povidone, gelatin and hydroxypropylcellulose, but the invention is not limited thereto.

This binder may be contained in an amount of 1 to 30 parts by weight, preferably 2 to 15 parts by weight, relative to 100 parts by weight of the active ingredient.

The diluent which is essentially contained in the sustained release tablet of an embodiment of the invention in addition to the hydrophilic polymer and the binder, may be either contained in the granules containing the active ingredient, or homogenously dispersed in the matrix of hydrophilic polymer. According to an embodiment of the invention, the diluent affects diffusion of the drug or decomposition of the hydrophilic polymer, and thus, the sustained release of the drug can be stably controlled in accordance with the properties of the diluent.

The diluent may be any material that is used in the field of pharmaceutics for the purpose, and may be selected from the group consisting of, for example, lactose, dextrin, starch, crystalline cellulose (e.g., Avicel™), calcium hydrogen phosphate, calcium carbonate, sugars and silicon dioxide, but the invention is not limited thereto.

The diluent may be contained in an amount of 5 to 60 parts by weight, preferably 10 to 30 parts by weight, relative to 100 parts by weight of the active ingredient.

The sustained release tablet of an embodiment of the invention may contain an enteric polymer as the binder or the diluent. When an enteric polymer is used as the binder or the diluent, it is possible to control the duration of release of the active ingredient as well as the release rate with time. The enteric polymer may be contained in the sustained release tablet of an embodiment of the invention in an amount of 3 to 30 parts by weight, preferably 5 to 20 parts by weight, relative to 100 parts by weight of the active ingredient. The enteric polymer may be any material which is insoluble at pH 5.0 or less, and becomes soluble at a pH ranging from 5.0 to 7.4. This enteric polymer allows controlling of the duration of release and release rate of the sustained release tablet of an embodiment of the invention, by means of the differences in the solubility according to the environmental pH.

Examples of the enteric polymer include acrylic resins such as acrylic latex dispersions, including Eudragit L, Eudragit S, Eudragit L-100-55-Rohm Pharma, and Eudragit L30D-Rohm Pharma; and other polymer such as cellulose acetate phthalate, polyvinyl acetate phthalate, and hydroxypropylmethylcellulose phthalate. Among these, a enteric polymer is Eudragit L-100-55. Both Eudragit L-100-55 in the form of fine powder, and Eudragit L30D in the form of aqueous dispersion are useful for an embodiment of the invention. These resins start to dissolve at about pH 5.5 or higher, and for this reason, they are advantageous in enhancing the drug release in the main area of the small intestine.

The sustained release tablet according to an embodiment of the invention may contain conventional, pharmaceutically acceptable additives in the granules or in the matrix, in addition to the binder, the hydrophilic polymer and the diluent, and examples of such additives include a filler, a lubricant, a gliding agent, a compression aid, and the like. For example, zinc stearate or magnesium stearate can be used as the gliding agent.

The sustained release tablet according to an embodiment of the invention may be coated, if necessary, with one of the numerous commercially available coating systems. When the tablet is coated, the taste of the medicament is masked, the tablet becomes easy to swallow, and in some cases, the external appearance of the dosage form can be improved. Such coating can be achieved by using sugar coating, which is well known in the art, or any one of numerous polymeric film coatings used in the formulation of medicaments.

Representative examples of the film coating agent include hydroxypropylmethylcellulose, carboxymethylcellulose, hydroxypropylcellulose, methylcellulose, ethylcellulose, acrylic resins, povidone, polyvinyl diethylaminoacetate, cellulose acetate phthalate, polyvinyl acetate phthalate, hydroxypropylmethylcellulose phthalate, acrylic latex emulsions, ethylcellulose latex emulsions, Pharmacoat (Shin-Etsu Chemical Co., Ltd.), Opadry (Colorcon, Inc.), and the like.

The release characteristic of the active ingredient of the sustained release tablet of an embodiment of the present invention can be controlled in accordance with the selection and contents of the hydrophilic polymer, binder and diluent contained in the sustained release tablet, and the like. For example, when the viscosity of the hydrophilic polymer increases, the release rate of the drug is decreased, or if the diluent is contained in the granule, the initial release rate may be increased. Furthermore, when an enteric polymer is used as the diluent or the binder, it is easy to maintain the duration of drug release for a longer time, and when the content of the diluent is increased, the release rate is also increased. Therefore, the drug release characteristics of the sustained release tablet of an embodiment of the invention can be specifically modified by altering the type and content of the hydrophilic polymer, binder or diluent, and the like.

The sustained release tablet of an embodiment of the invention can be prepared by a method comprising the steps of: preparing granules containing zaltoprofen and a binder; and tabletting the mixture after mixing the granules with a hydrophilic polymer and pharmaceutically acceptable additives, in which a diluent is added during the step of preparing granules or during the step of tabletting.

In the method described above, the step of preparing granules can be performed by a conventional method of granulation known in the field of pharmaceutics, using zaltoprofen as the active ingredient, a binder, optionally a diluent, and additives that are conventionally used for granulation in the field of pharmaceutics. The granules thus prepared can be mixed with a hydrophilic polymer, optionally a diluent, and additives that are used for granulation in the field of pharmaceutics, such as a gliding agent, and the resultant mixture can be tableted to yield the sustained release tablet of an embodiment of the present invention in the form of a matrix.

### EXAMPLES

Hereinafter, embodiments of the present invention will be described in more detail with reference to Examples. However, these Examples are for the illustrative purpose only, and the invention is not limited by these Examples.

### Examples 1 and 2

A binder solution was prepared by dissolving povidone in a 20% aqueous ethanol solution, and the prepared binder solution was added to zaltoprofen powder, the active ingredient. The mixture was subjected to granulation using a 30-mesh screen, and the granules thus formed were dried at 40° C. for 2 hours. The obtained granules were mixed with hydroxypropylmethylcellulose, which is a hydrophilic polymer, crystalline cellulose, silicon dioxide and magnesium stearate, and the mixture was tableted to give a sustained release tablet. The contents of the respective components constituting the respective sustained release tablets are indicated in Table 1 below.

### Example 3

A sustained release tablet was prepared in the same method as in Example 2, except that the crystalline cellulose used after the process of preparing granules in Example 2 was added during the process of preparing granules at this time. The contents of the respective components constituting the sustained release tablet are indicated in Table 1 below.

TABLE 1
Composition of Tablet

| | Example 1 | Example 2 | Example 3 |
|---|---|---|---|
| Component | (unit: mg) | | |
| Zaltoprofen | 240 | 240 | 240 |
| Polyvidon K30 | 7.9 | 7.9 | 7.9 |
| Avicel PH101 | | | 37 |
| Methocel K4M CL (4000 cps) | 70 | | |
| Methocel K100LV (100 cps) | | 70 | 70 |
| Avicel PH102 | 37 | 37 | |
| SiO₂ | 3.5 | 3.5 | 3.5 |
| S—Mg | 1.6 | 1.6 | 1.6 |
| Tablet weight | 360 | 360 | 360 |

### Example 4

After mixing zaltoprofen with crystalline cellulose, a binder solution prepared by completely dissolving Eudragit S100 in an ethanol solution was used to prepare granules in the same manner as in Example 1 above. The obtained granules were mixed with hydroxypropylmethylcellulose (100 cps), crystalline cellulose, silicon dioxide and magnesium stearate, and the mixture was tableted to give a sustained release tablet. The contents of the respective components constituting the sustained release tablet are indicated in Table 2 below.

TABLE 2
Composition of Tablet

| Component | Example 4 (unit: mg) |
|---|---|
| Zaltoprofen | 240 |
| Avicel PH101 | 22 |
| Eudragit S100 | 20 |
| Methocel K100LV | 80 |
| Avicel PH102 | 30 |
| SiO₂ | 4 |
| S—Mg | 4 |
| Tablet weight | 400 |

### Example 5

After mixing zaltoprofen with crystalline cellulose, a binder prepared by adding polyethylene glycol 400 to a povidone binder was used to prepare granules in the same manner as in Example 1. The prepared granules were mixed with carbomer, crystalline cellulose, silicon dioxide and magnesium stearate, and the mixture was tableted to give a sustained release tablet. The contents of the respective components constituting the sustained release tablet are indicated in Table 3 below.

### Example 6

After mixing zaltoprofein with Eudragit L-100-55, which is an enteric polymer, a binder prepared by adding polyethylene glycol 400 to a povidone binder was used to prepare granules in the same manner as in Example 1. The prepared granules were mixed with carbomer, crystalline cellulose, silicon dioxide and magnesium stearate, and the mixture was tableted to give a sustained release tablet. The contents of the respective components constituting the sustained release tablet are indicated in Table 3 below.

### Example 7

Unlike Example 6, the active ingredient zaltoprofen was mixed with crystalline cellulose, and then an aqueous dispersion of Eudragit L30D was used as the binder to prepare granules in the same manner as in Example 1. The prepared granules were mixed with carbomer, silicon dioxide and magnesium stearate, and the mixture was tableted to give a sustained release tablet. The contents of the respective components constituting the sustained release tablet are indicated in Table 3 below.

TABLE 3
Composition of Tablet

| | Example 5 | Example 6 | Example 7 |
|---|---|---|---|
| Component | (unit: mg) | | |
| Zaltoprofen | 240 | 240 | 240 |
| Avicel PH101 | 48 | | 37 |
| Eudragit L00-55 | | 48 | |
| Eudragit L30D-55 | | | 100(30) |
| Polyvidon K30 | 6.7 | 6.7 | |
| PEG 400 | 6.7 | 6.7 | |
| Carbopol 71G | 76.6 | 76.6 | 77.0 |
| Avicel PH102 | 16.0 | 16.0 | |
| SiO₂ | 2.0 | 2.0 | 3.0 |
| S—Mg | 4.0 | 4.0 | 3.0 |
| Tablet weight | 400 | 400 | 390 |

### Examples 8 to 10

After mixing the active ingredient zaltoprofen with crystalline cellulose and Eudragit L100 which is an enteric polymer, a povidone binder was used to prepare granules in the same manner as in Example 1. The prepared granules were mixed with carbomer, crystalline cellulose, silicon dioxide and magnesium stearate, and the mixture was tableted to produce sustained release tablets of Examples 8 to 10. The contents of the respective components constituting the respective sustained release tablets are indicated in Table 4 below.

TABLE 4
Composition of Tablet

| | Example 8 | Example 9 | Example 10 |
|---|---|---|---|
| Component | (unit: mg) | | |
| Zaltoprofen | 240 | 240 | 240 |
| Avicel PH101 | 24 | 24 | 24 |
| Eudragit L100-55 | 12 | 15 | 12 |
| Polyvidon K30 | 7.27 | 7.5 | 7.27 |
| Carbopol 71G | 78 | 65 | 60 |
| Avicel PH102 | 32 | 41.7 | 50 |
| SiO₂ | 3.4 | 3.4 | 3.4 |
| S—Mg | 3.4 | 3.4 | 3.4 |
| Tablet weight | 400.07 | 400 | 400.07 |

### Experimental Example

1) Test Method

A dissolution test was conducted on the sustained release tablets prepared in Examples 1 through 10.

Each of the sustained release tablets prepared in Examples 1 to 10 was subjected to a dissolution test under the following conditions according to a paddle method, and the dissolution rate of the active ingredient, zaltoprofen, from each of the sustained release tablets was measured over time.

The test conditions used in the dissolution test are as follows.

Eluent: Phosphate buffer solution at pH 6.5 (0.05 M potassium dihydrogen phosphate, 0.05 M sodium hydrogen phosphate, and adjusted to pH 6.5 using phosphoric acid)

Temperature of eluent: 37° C.±0.5° C.

Amount of eluent: 900 ml

Stirring speed: 100 rpm

Sampling: An amount of the eluent was taken at every sampling time, filtered through a filter, and used as the analyte. The same amount of fresh eluent was added to compensate for the amount taken for sampling.

The dissolution rate of the active ingredient was analyzed using the analyte obtained at every sampling time for the dissolution test. The analysis of dissolution rate was performed by liquid chromatography under the following conditions.

Detector: Ultraviolet absorption spectrometer (measurement wavelength: 240 nm)

Column: Capcell Pak (ODS, 5 μm, 4×150 mm, Shisheido)

Mobile phase: Acetonitrile:water:glacial acetic acid (300:200:1)

Flow rate: 1 ml/min

2) Test Results

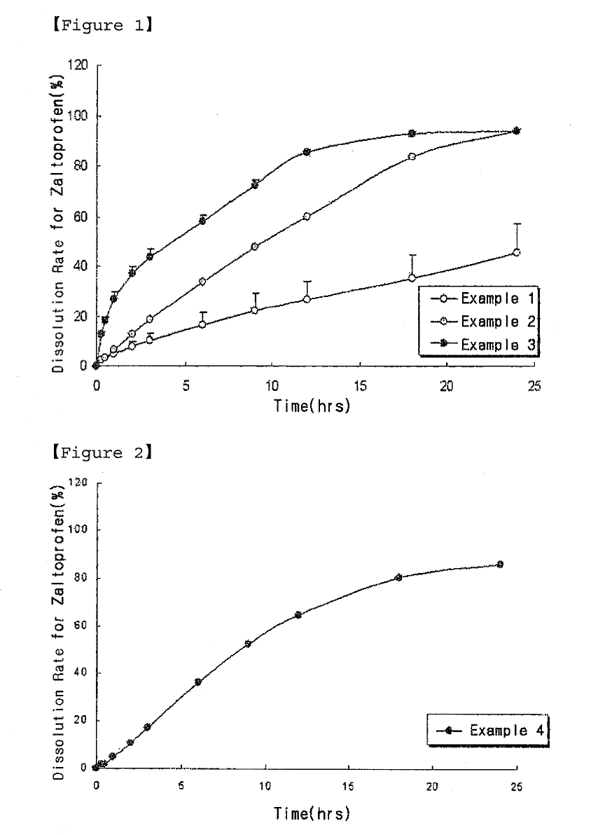

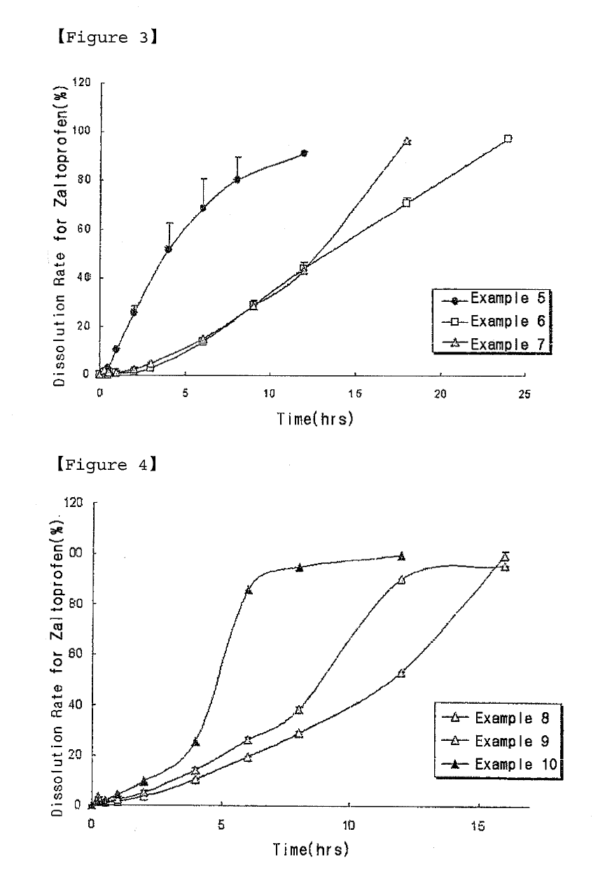

The results of measuring the dissolution rate over time are presented in FIG. 1 (Examples 1 to 3), FIG. 2 (Example 4), FIG. 3 (Examples 5 to 7) and FIG. 4 (Examples 8 to 10).

Upon examining the dissolution rates over time in FIG. 1 to FIG. 4, all of the sustained release tablets in general exhibit the characteristic of continuously releasing the active ingredient for 12 hours to 24 hours. Thus, it was confirmed that the sustained release tablets of an embodiment of the present invention were sustained release tablets suitable for the desired once-daily administration.

The dissolution patterns which varied from Example to Example depending on the differences in specific components were analyzed.

Referring to FIG. 1, it was confirmed from the results of the dissolution test that the sustained release tablet of Example 1 which was prepared using a hydroxypropylmethylcellulose with a viscosity of 4000 cps, and the sustained release tablet of Example 2 prepared using a hydroxypropylmethylcellulose with a viscosity of 100 cps, showed dissolution rates of 40% and 80%, respectively, in 24 hours, and this difference was due to the difference in the viscosity. Further, the sustained release tablet of Example 3, which was prepared in the same manner as in Example 2, except that the diluent crystalline cellulose was added during the process of preparing granules of the active ingredient, showed an increase in the initial dissolution rate compared with the sustained release tablet of Example 2.

Therefore, it can be seen from the test results shown in FIG. 1 that when the viscosity of the hydrophilic polymer increases, the release rate of the active ingredient is decreased, and that in the case where the diluent is present in the granules, the initial dissolution rate can be increased. Furthermore, even for sustained release tablets having the same composition, a sustained release tablet of zaltoprofen which is capable of controlling the dissolution rate can be prepared by varying the process for preparation.

Referring to FIG. 3, the dissolution test results of the zaltoprofen sustained release tablets prepared in Examples 5 to 7 showed that the sustained release tablet of Example 5 in which crystalline cellulose was used as the diluent in the process of preparing granules of the active ingredient, resulted in a dissolution rate of 90% or greater in 12 hours, while the sustained release tablet of Example 6 in which an enteric polymer was used as the diluent, resulted in a dissolution rate of 100% in 24 hours. The sustained release tablet of Example 7 in which Eudragit L30D supplied as an aqueous dispersion was used as the binder, showed a dissolution rate of 100% in 18 hours. Therefore, it was confirmed that it was easy to delay and control the drug release by varying the type of the binder and the diluent.

According to FIG. 4 showing the dissolution test results for Examples 8 to 10, it was confirmed that a decrease in the content of the hydrophilic polymer or an increase in the content of the diluent resulted in an increase in the release rate of the drug.

It can be seen from the test results that the dissolution characteristics of the sustained release tablet according to an embodiment of the present invention can be easily controlled by appropriately selecting the types and contents of the hydrophilic polymer, binder and diluent contained in the sustained release tablet.

As discussed above, according to an embodiment of the present invention, there can be provided a zaltoprofen-containing sustained release tablet which can continuously release the active ingredient so that once-daily administration is made possible, and whose dissolution characteristics can be easily controlled by varying the type and content of the hydrophilic polymer, binder or diluent contained in the sustained release tablet, and a method for preparing the same.

What is claimed is:

1. A zaltoprofen-containing sustained release tablet comprising granules containing zaltoprofen and a binder, wherein the granules are dispersed in a matrix comprising a hydrophilic polymer, wherein the tablet further comprises a diluent that is present in the granules, or in the matrix.

2. The sustained release tablet according to claim 1, wherein 5 to 60 parts by weight of the hydrophilic polymer, 1 to 30 parts by weight of the binder, and 5 to 60 parts by weight of the diluent are contained in the tablet, relative to 100 parts by weight of zaltoprofen.

3. The sustained release tablet according to claim 1, wherein the hydrophilic polymer has a viscosity of 5 to 100,000 cps.

4. The sustained release tablet according to claim 1, wherein the hydrophilic polymer is selected from the group consisting of acacia gum, tragacanth gum, locust bean gum, guar gum, Karaya gum, agar-agar, pectin, carrageenan, soluble or insoluble alginates, methylcellulose, hydroxypropylmethylcellulose, carbomer, hydroxypropylcellulose, hydroxyethylcellulose, sodium carboxymethylcellulose, carboxypolymethylene, gelatin, casein, zein, bentonite, a natural or synthetic, anionic or nonionic hydrophilic rubber, modified cellulose-based materials, and proteinaceous materials.

5. The sustained release tablet according to claim 1, wherein the binder is selected from the group consisting of povidone, gelatin, hydroxypropylcellulose, hydroxypropylmethylcellulose, and an enteric polymer.

6. The sustained release tablet according to claim 1, wherein the diluent is selected from the group consisting of lactose, dextrin, starch, crystalline cellulose, an enteric polymer, calcium hydrogen phosphate, calcium carbonate, sugars and silicon dioxide.

7. The sustained release tablet according to claim 4, wherein the enteric polymer is selected from the group consisting of Eudragit L, Eudragit S, Eudragit L-100-55-Rohm Pharma, Eudragit L30D-Rohm Pharma, cellulose acetate phthalate, polyvinyl acetate phthalate, and hydroxypropylmethylcellulose phthalate.

8. The sustained release tablet according to claim 5, wherein the enteric polymer is selected from the group consisting of Eudragit L, Eudragit S, Eudragit L-100-55-Rohm Pharma, Eudragit L30D-Rohm Pharma, cellulose acetate phthalate, polyvinyl acetate phthalate, and hydroxypropylmethylcellulose phthalate.

9. A method of preparing the sustained release tablet according to claim 1, comprising:
providing granules containing zaltoprofen and the binder;
mixing the granules with a hydrophilic polymer and pharmaceutically acceptable additives so as to provide a mixture; and
shaping the mixture into a tablet.

10. The method of claim 9, wherein the diluent is contained in the granules.

11. The method of claim 9, wherein mixing comprises adding the diluent so as to include the diluent in the mixture.
